(19) United States
(12) Patent Application Publication
(10) Pub. No.: US 2007/0088286 A1
(43) Pub. Date: Apr. 19, 2007

(54) Identification system for intravenous tubing

(76) Inventor: Arnold M. Brier, North Oaks, MN (US)

Correspondence Address: SHUMAKER & SIEFFERT, P. A., 8425 SEASONS PARKWAY, SUITE 105, ST. PAUL, MN, 55125, US

(21) Appl. No.: 11/498,542

(22) Filed: Aug. 3, 2006

Related U.S. Application Data
(60) Provisional application No. 60/704,979, filed on Aug. 3, 2005.

(51) Int. Cl. A61M 5/00

(52) U.S. Cl. 604/189

(57) ABSTRACT

In general, the invention relates to an identification system for IV tubing. More particularly, colored patterned flags are adhered to IV lines, and matching patterned, colored labels are adhered to the reservoirs that attach to the lines. Each line and its corresponding reservoir are marked with at least one flag and label that have a unique color and pattern such that all of the flags and labels on one line and its corresponding reservoir match but differ in both color and pattern to all of the flags and labels on the other lines and reservoirs.

This application claims the benefit of U.S. Provisional Application No. 60/704,979, filed Aug. 3, 2005, which is incorporated herein by reference in its entirety.

### TECHNICAL FIELD

The invention relates to a system and method for rapid and accurate identification of the materials administered to a patient via intravenous (IV) tubing.

### BACKGROUND

FIGS. 1A-1C illustrate a typical patient undergoing medical treatment in, for example, an Intensive Care or Skilled Care Unit, a nursing home, a medical office, or under emergency conditions in an ambulance, a helicopter or in the field. Numerous medical tubing and other lines may be connected to the patient to provide various types of treatment and/or therapy. In FIG. 1A, IV lines 8 are shown on a patient 10. Additionally electrical wires 12 leading to a cardiac monitor (not shown) are taped to the patient 10. In FIG. 1B, IV lines 14 are connected to a patient 16. Additionally, a blood pressure cuff 18 and additional medical tubing 20 are used to monitor the patient 16. FIG. 1C shows reservoir bags 22A-22C on an IV pole 24. Pumps 26A-26C regulate flow rate from the reservoir bags 22A-22C to a patient (not shown) and are also located on the IV pole 24. IV tubing 28A-28C travels from reservoir bags 22A-22C to pumps 26A-26C and then to a patient. In addition to the equipment shown in FIGS. 1A-1C, the patient may have a breathing tube in the trachea attached to a respirator at bedside, an electric oxygen monitor on a finger, blood pressure monitors attached to the patient from overhead, a urinary catheter running from the bladder to a collection reservoir where kidney function can be monitored, and a variety of other medical lines connected to his or her body. Often tubes are inserted into the stomach through the nose or the abdominal wall to administer liquid nutrition. Depending on the patient's illness, drainage tubes may be in place to drain body fluids from the head, chest, abdomen or rectum. Each drainage tube is connected to its own measuring reservoir.

A patient's treatment program almost always includes IV infusions of at least one liquid material such as, for example, drugs, electrolytes and the like. These liquid materials are segregated from each other in individual small plastic IV reservoir bags and are administered at frequent intervals or continuously during a typical hospital day. Since the liquids are kept separate, each bag is suspended from an IV pole and typically connected by flexible, clear, narrow-caliber IV tubing via a small electric pump (used to regulate flow rate for that particular liquid) to an IV hub at the patient. Multiple IV lines enter the patient at the IV hub. Since each liquid material has its own IV tubing and pump, and may be in either constant or intermittent connection with the patient's IV hub, there is often a “spaghetti-like” mass of clear IV tubing attached to the patient.

### SUMMARY

Since the patient may be connected to large numbers of tubes and wires, and several types of medical equipment, and there may be multiple IV hub sites where various liquid materials are delivered to the patient, identifying which IV tubing contains a specific drug or other liquid material can be very difficult and time consuming.

Currently, identification of each line is particularly difficult and time-consuming because of all the IV lines are made of identical clear plastic tubing with the only indicator contents being a small printed label on the reservoir bag.

The mass of clear IV lines are difficult to distinguish, which requires additional treatment time. Often the medical professional must identify the bag containing a particular drug or other liquid material and trace the tubing manually back to the patient through the jumble of IV tubes. Many times the reverse is necessary: the nurse or other medical professional must follow an individual line from the patient back up to the reservoir bag to ascertain which drug or other liquid material is flowing through the line. Adding to the difficulty, the labels on the reservoir bag are often in small print and hard read.

The lack of labeling on the multitude of tubing and other lines in FIGS. 1A-1C is highly inefficient, which increases treatment time. The lack of rapidly identifiable labels on the IV lines also increases the likelihood that a medical professional may mistakenly select an incorrect line during treatment, particularly in an emergency situation.

An IV labeling system could simplify the process of identifying the contents of each IV line. Such a labeling system could potentially reduce treatment time and reduce and/or eliminate medical errors.

In general, the invention relates to an identification system for IV tubing. More particularly, colored patterned flags are adhered to IV lines, and matching patterned, colored labels are adhered to the reservoirs that attach to the lines. Each line and its corresponding reservoir are marked with at least one flag and label that have a unique color and pattern such that all of the flags and labels on one line and its corresponding reservoir match but differ in both color and pattern to all of the flags and labels on the other lines and reservoirs.

In one embodiment, the invention is directed to a system comprising a plurality of IV lines, wherein each line delivers a fluid from a corresponding reservoir to a patient; at least one colored flag on at least one portion of one of the lines; and a colored label on the corresponding reservoir, wherein the at least one flag and the label are the same color.

In another embodiment, the invention is directed to a method of identifying a plurality of IV lines, wherein each line delivers a fluid from a corresponding reservoir to a patient, comprising placing at least one colored flag on at least one portion of one of the lines; and placing a colored label on the corresponding reservoir, wherein the at least one flag and the label are the same color.

In another embodiment, the invention is directed to a kit for a labeling system for IV lines, wherein each line delivers a fluid from a corresponding reservoir to a patient, comprising a dispenser containing a plurality of sheets, wherein at least one colored adhesive-backed flag for application to an IV line and at least one matching colored adhesive-backed label for application to a corresponding reservoir are adhered to each sheet, wherein each sheet contains a different color of the at least one flag and the at least one matching label, and wherein the at least one flag and the at least one label may be easily peeled off each sheet.

The labeling system described in this disclosure may allow a medical professional such as, for example, a nurse, doctor or paramedic, to make rapid, accurate visual identification of each IV line at the site where it enters the patient. A system that allows medical personnel to immediately associate an IV line with the drug or other liquid material it contains would save time and improve efficiency.

The medical professional's continuous surveillance of the many devices and lines attached to the patient is frequently interrupted by the need to administer IV therapies for specific periods of time. The safe management of this forest of medical equipment attached to the patient puts a premium on simplification when the medical staff has a heavy workload as well as in emergency situations. Such a system reduces the probability of medical errors such as combining incompatible drugs in the same IV line or setting the incorrect infusion rate at the pump because of misidentification of the IV line. Errors in hospital care are now recognized as a major case of bad medical outcomes and excessive medical costs. Simplification of medical care is critical in reducing such medical errors.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A-1C are illustrations of a typical patient in an Intensive Care or Skilled Care Unit and the numerous amount of medical tubing and other lines that may be connected to a hospital patient.

FIG. 2 is an illustration of a patient with multiple IV lines that are labeled according to one embodiment of the invention.

FIGS. 3A-3C are illustrations of a kit for a labeling system for IV lines and sheets that may be included as part of the kit.

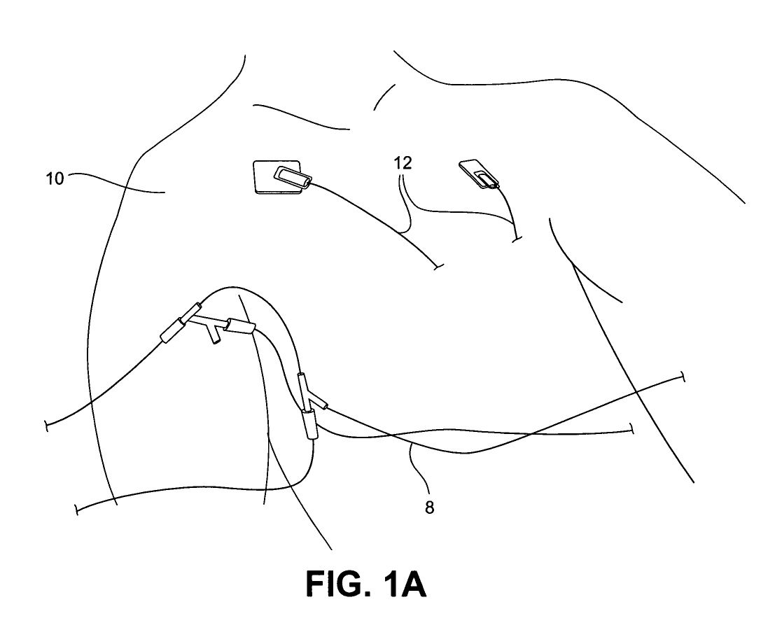

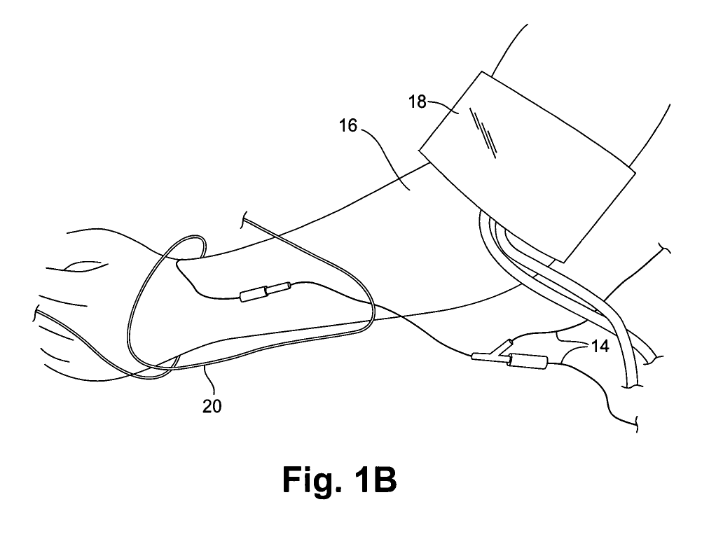

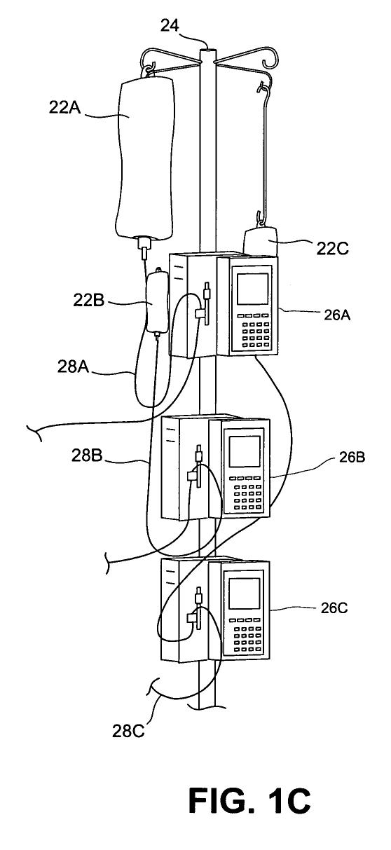

### DETAILED DESCRIPTION

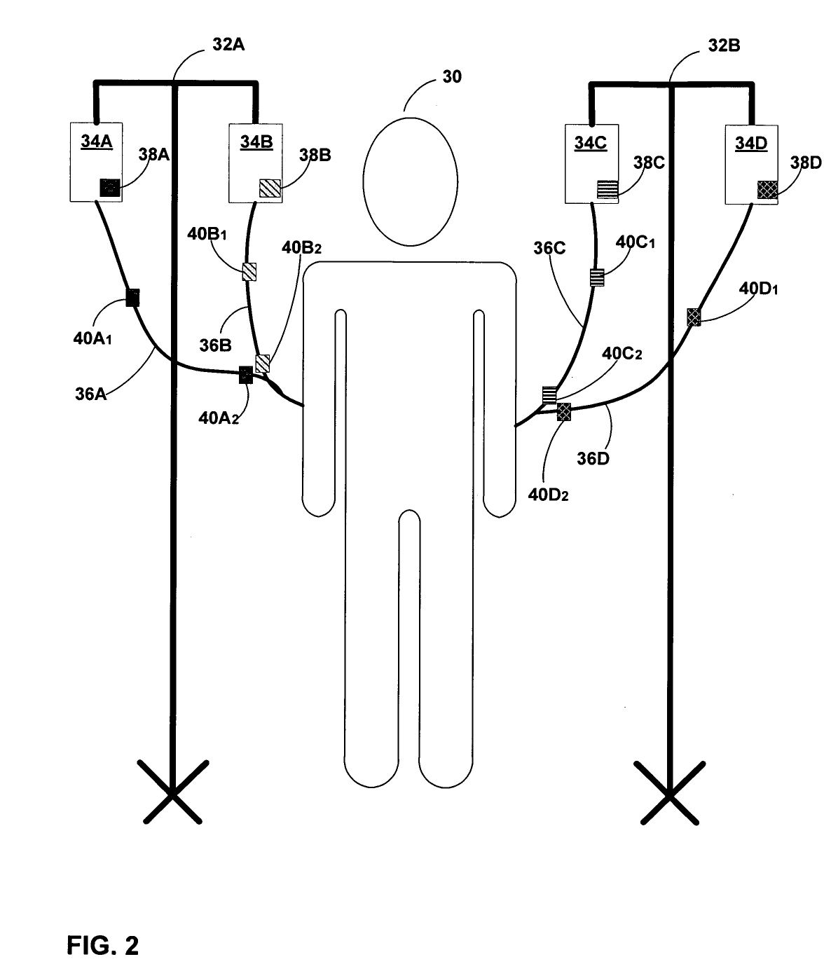

FIG. 2 is an illustration of a patient with multiple IV lines that are labeled according to one embodiment of the invention. IV lines 36A-36D (collectively IV lines 36) are connected to patient 30 and respective reservoir bags 34A-34D (collectively reservoir bags 34). Reservoir bags 34A and 34B are located on IV pole 32A, and reservoir bags 34C and 34D are located on IV pole 32B. IV lines 36 each contain a pump (not shown) located on IV lines 36 between reservoir bags 34 and patient 30. Reservoir bags 34 contain labels 38A-38D (collectively labels 38). For example, reservoir 34A contains label 38A. Additionally, each IV line contains at least one flag. For example, IV line 36A contains flags 40A₁ and 40A₂. Label 38A and flags 40A₁ and 40A₂ all contain the same color and pattern. The matching labels and flags on each line contain a color and pattern unique to that line. For example, label 38A and flags 40A₁ and 40A₂ on line 36A do not have the same color or pattern as label 38B and flags 40B₁ and 40B₂ on line 36B or any other labels and flags on any additional lines. Identification by both color and pattern is a safety redundancy. The colors and patterns may be distinct and bright, possibly fluorescent for enhanced visibility in low light environments.

With the labeling system in use, the nurse or other medical professional will see multiple IV lines 36, each easily identified by its special flagging (flags 40A₁-40D₁ and 40A₂-40D₂, collectively flags 40), and may quickly confirm the contents of each line by comparing flags 40 with matching label 38 on reservoir bag 34.

Labels 38 and flags 40 may be adhesive-backed such that labels 38 and flags 40 adhere securely to IV lines 36 without an additional adhesive. Additionally, flags 40 may be located at regular intervals along IV lines 36. In another embodiment, each line may contain only one flag near the site where the IV enters the patient. As an example, IV line 36A may only contain flag 40A₂ and not both flags 40A₁ and 40A₂. Multiple IV lines often enter the patent at one location, which adds to the difficulty of determining which IV line contains which fluid. As illustrated in FIG. 2, IV lines 36A and 36B enter the patient 30 at the same location. In the preferred embodiment, each IV line contains a flag near the site where the IV line enters the patient and may also contain additional flags spaced at regular intervals along the IV line.

Additionally, an open area of the labels 38 may be used to identify the contents of the reservoir bag. The name of the fluid, such as a drug name, may be handwritten in the open area.

Alternatively a smaller adhesive label, referred to herein as a “sticker,” may be used to identify a fluid in a reservoir. Identification information, such as a drug name, may be printed on the stickers. The pharmacy may print the identification information on a sticker when it prepares the IV reservoir bag. The pharmacy may print on the stickers using a bold type, making the stickers easier to read. A pre-printed, bold-faced sticker identifying a fluid in a reservoir may accompany the reservoir bag when it is delivered from the pharmacy to the nurse or other medical professional.

At bedside, the medical professional may attach the printed sticker to the colored label selected for a reservoir and affix the completed label to the reservoir bag. When the identification sticker is assembled and attached, the identification information may be easily read and clearly surrounded by the selected color and pattern of the label.

The number, size, and shape of flags 40 and labels 38 were chosen for illustrative purposes, and these may be modified as necessary for a particular application. Any number of flags may also be placed on each IV line, and multiple labels may be placed on each reservoir. Additionally, the labeling system may be used on any number of IV lines or in combination with any other medical equipment.

The presently described identification system is simple to adopt, intuitive in its use and requires no special training to install. It can be produced using available materials and technology. Thus, the system should be inexpensive to manufacture and may benefit hospitals and other healthcare facilities at minimal expense. Additionally, since the identification system improves the safety and efficiency of IV therapy, a core component of medical care, and is easily understood and applied with no language barrier, this system has the potential for use in healthcare facilities world wide.

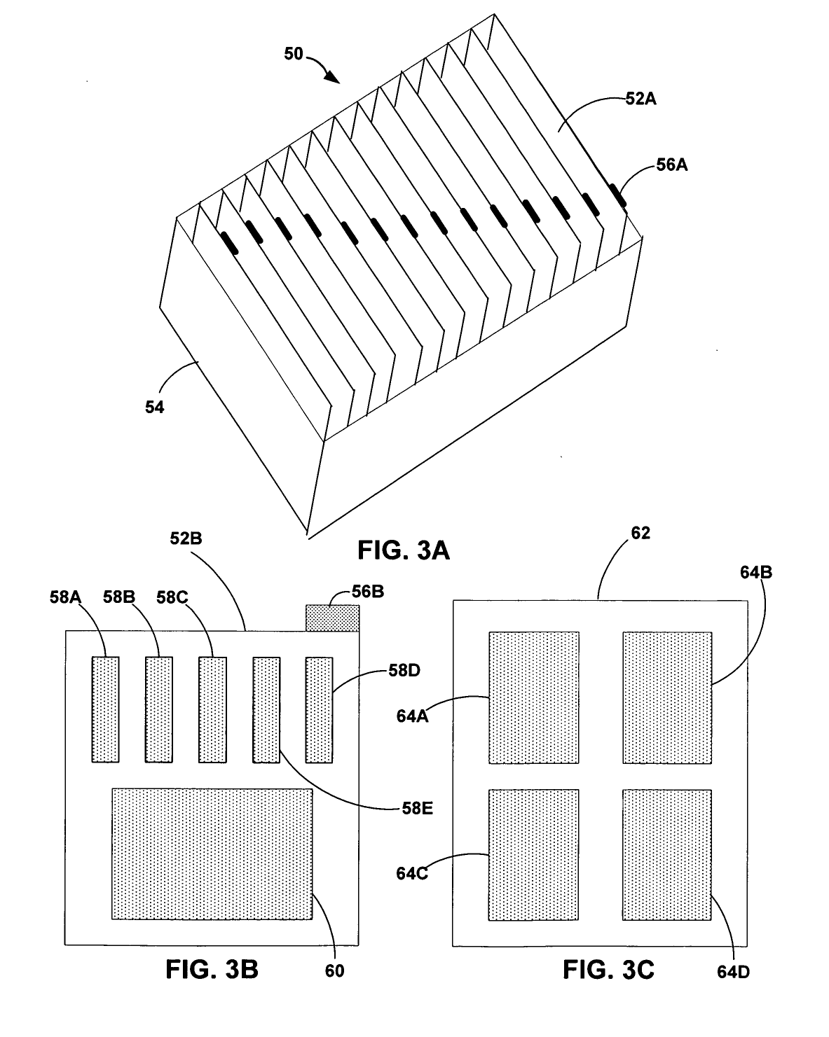

FIGS. 3A-3C show a kit for a labeling system for IV lines and sheets that may be included as part of the kit. In FIG. 3A, kit 50 contains a dispenser 54 full of labeling sheets (reference 52A identifies a labeling sheet). Dispenser 54 may be easily stored in each patient room to facilitate the nurse's use of the labeling system according to the invention. Each sheet may contain a tab that extends from dispenser 54 (reference 56A identifies a tab). The configuration of dispenser 54 may be changed in any manner as long as the tabs extend from the dispenser. In a preferred embodiment, the sheets are held upright by the dispenser with their colored tabs sticking up so that the medical professional can easily finger through the sheets using the tabs.

FIG. 3B shows a detailed view of labeling sheet 52B. Labeling sheet 52B contains multiple flags 58A-58F (collectively flags 58) for application to an IV line and label 60 for application to a corresponding reservoir. Labeling sheet 52B may contain any number of flags 58 and also may include more than one label 60. Flags 58, label 60, and tab 56B on labeling sheet 52B match in both color and pattern. Using both color and pattern as identifying characteristics on flags 58, label 60, and tab 56B is a safety redundancy. The colors and patterns may be distinct and bright, possibly fluorescent for enhanced visibility in low light environments.

Each additional sheet contains a matching label, tab, and flags and has similar characteristics of detailed sheet 52B. However, not every sheet contains the same color and pattern as sheet 52B. A variety of sheets are included to provide a multitude of color and pattern choices. Having a matching tab that extends from the dispenser on each sheet may allow the medical professional to quickly grasp the tab of the desired color and pull out the sheet needed. The medical professional may also identify the sheet by pattern. Offering a full complement of sheets in a variety of colors and patterns may aid the medical professional's selection of colors and patterns that are not already in use.

Label 60 and flags 58 may be adhesive-backed such that label 60 and flags 58 adhere securely to the reservoir and IV line without additional adhesive. Additionally, label 60 and flags 58 may be easily peeled off of sheet 52B. The adhesive may be selected to allow for easy peeling from sheet 52B and also secure adhesion to plastic IV tubing. Sheet 52B may be firm enough to make handling and peeling easy. The additional sheets supplied in kit 50 of FIG. 3A may also have these characteristics.

When an IV fluid arrives in its plastic reservoir bag and is attached to new IV tubing to be connected to the patient, the medical professional will select a sheet of flags and matching labels (such as sheet 52B). The label will be affixed to the reservoir bag. Additionally, an open area of the label may accommodate a means for identifying the contents of the reservoir bag. The name of the fluid, such as a drug name, may be handwritten in the open area. Alternatively a sticker may be provided.

Kit 50 of FIG. 3A may provide a supply of blank stickers of custom size and shape. The blank stickers may be located on multiple sticker sheets. FIG. 3C depicts sheet 62 of blank stickers 64A-64D (collectively stickers 64). Stickers 64 can be used to identify a fluid in a reservoir. Identification information, such as a drug name, may be printed on stickers 64. The pharmacy may print the identification information on a sticker when it prepares the IV reservoir bag. The pharmacy may print on stickers 64 using a bold type, making stickers 64 easier to read. Also, sheet 62 may be fed into a printer. The pre-printed, bold-faced sticker identifying the fluid in the reservoir may accompany the reservoir bag when it is delivered from the pharmacy to the medical professional.

At bedside, the medical professional may attach the printed sticker to the colored label selected for a reservoir and affix the completed label to the reservoir bag. When the identification sticker is assembled and attached, the fluid name may be easily read and clearly surrounded by the selected color and pattern of the label.

Various embodiments of the invention have been described. These and other embodiments are within the scope of the following claims.

1. A system comprising:
a plurality of intravenous (IV) lines, wherein each line delivers a fluid from a corresponding reservoir to a patient;
at least one colored flag on at least one portion of one of the lines; and
a colored label on the corresponding reservoir, wherein the at least one flag and the label are the same color.

2. The system of claim 1, wherein the at least one colored flag further comprises a pattern; and the colored label further comprises a pattern, wherein the at least one flag and the label have the same pattern.

3. The system of claim 2, wherein the colored flags and the colored label are adhesive-backed.

4. The system of claim 3, further comprising at least one colored flag on at least one portion of each of the plurality of lines; and a matching colored label on each of the corresponding reservoirs, wherein the at least one flag on a first line differs both in pattern and color from at least one flag on a second line.

5. The system of claim 4, wherein the flags are located on multiple portions of each of the plurality of the lines, and wherein the flags are spaced at predetermined intervals along each line.

6. The system of claim 4, wherein at least one colored flag on each of the plurality of lines is located near a site where the line enters the patient.

7. The system of claim 4, further comprising an identification sticker on each label, wherein the identification sticker identifies the contents of a reservoir on which the label is placed, and wherein the pattern and color of the label are visible when the identification sticker is attached.

8. A method of identifying a plurality of intravenous (IV) lines, wherein each line delivers a fluid from a corresponding reservoir to a patient, comprising:
placing at least one colored flag on at least one portion of one of the lines; and
placing a colored label on the corresponding reservoir, wherein the at least one flag and the label are the same color.

9. The method of claim 8, wherein the at least one colored flag further comprises a pattern; and the colored label further comprises a pattern, wherein the at least one flag and the label have the same pattern.

10. The method of claim 9, wherein the colored flags and the colored labels are adhesive-backed.

11. The method of claim 10, further comprising at least one colored flag on at least one portion of each of the plurality of lines; and a matching colored label on each of the corresponding reservoirs, wherein the at least one flag on a first line differs both in pattern and color from at least one flag on a second line.

12. The method of claim 11, wherein the flags are located on multiple portions of each of the plurality of the lines, and wherein the flags are spaced at predetermined intervals along each line.

13. The method of claim 11, wherein at least one colored flag on each of the plurality of lines is located near a site where the line enters the patient.

14. The method of claim 11, further comprising an identification sticker on each label, wherein the identification sticker identifies the contents of a reservoir on which the label is placed, and wherein the pattern and color of the label are visible when the identification sticker is attached.

15. A kit for a labeling system for intravenous (IV) lines, wherein each line delivers a fluid from a corresponding reservoir to a patient, comprising:
a dispenser containing a plurality of sheets,
wherein at least one colored adhesive-backed flag for application to an IV line and at least one matching colored adhesive-backed label for application to a corresponding reservoir are adhered to each sheet, wherein the plurality of sheets contain a variety of colors of the at least one flag and the at least one matching label, and wherein the at least one flag and the at least one label may be easily peeled off each sheet.

16. The kit of claim 15, wherein the at least one colored flag further comprises a pattern; and the at least one colored label further comprises a pattern, wherein the at least one flag and the at least one label have the same pattern.

17. The kit of claim 16, wherein each sheet further comprises a colored tab that extends from the dispenser, wherein the colored tab matches the color of the at least one flag and at least one label on the sheet.

18. The kit of claim 16, wherein each sheet further comprises a colored, patterned tab that extends from the dispenser, wherein the colored, patterned tab matches the color and the pattern of the at least one flag and at least one label on the sheet.

19. The kit of claim 15, further comprising a plurality of identification sticker sheets, wherein each identification sticker sheet comprises a plurality of identification stickers for application to the colored labels, wherein the identification stickers are smaller than the colored labels such that the color of the label is visible when the identification sticker is attached.
